(19) United States
(12) Patent Application Publication
(10) Pub. No.: US 2022/0192867 A1
(43) Pub. Date: Jun. 23, 2022

(54) Targeted Temperature Management Systems, Pads, and Methods Thereof

(71) Applicant: C. R. Bard, Inc., Franklin Lakes, NJ (US)

(72) Inventors: Madeline Stich, Thornton, CO (US); Gabriel A. Johnston, Broomfield, CO (US); Sean E. Walker, Platteville, CO (US); Rebecca D. Salisbury, Lafayette, CO (US); James David Hughett, SR., Monroe, GA (US); Michael R. Hoglund, Windsor, CO (US)

(21) Appl. No.: 17/552,309

(22) Filed: Dec. 15, 2021

Related U.S. Application Data
(60) Provisional application No. 63/130,287, filed on Dec. 23, 2020.

(51) Int. Cl. A61F 7/02; A61F 7/08

(52) U.S. Cl. CPC A61F 7/02; A61F 2007/0029; A61F 7/08

(57) ABSTRACT

Disclosed herein are systems, pads, and methods thereof for targeted temperature management. A first pad can include a multilayered pad body and an inflatable cuff around the pad body. The pad body can include a conformable layer, a conduit layer over the conformable layer, and an insulation layer over the conduit layer. The conformable layer can include a conformable material configured to conform around a limb of a patient. The conduit layer can include one or more conduits configured to convey a fluid through the conduit layer. The insulation layer can be configured to insulate the conduit layer from an ambient temperature of an environment around the patient. The inflatable cuff can be configured to press the pad body against the limb of the patient when the inflatable cuff is inflated. Methods of the systems and pads can include methods of use.

### PRIORITY

This application claims the benefit of priority to U.S. Provisional Patent Application No. 63/130,287, filed Dec. 23, 2020, which is incorporated by reference in its entirety into this application.

### BACKGROUND

Current systems for target temperature management (“TTM”) require adhesive pads adhered onto patients in order to maintain sufficient contact between the pads and the patients for inducing therapeutic hypothermia by circulating chilled fluid through the pads. However, such pads can cause skin irritation from the adhesive of the pads. In addition, clinicians often require open access to the patients for medical procedures. The adhesive pads can hinder the open access required by the clinicians.

Disclosed herein are TTM systems, pads, and methods thereof that address the foregoing.

### SUMMARY

Disclosed herein is a pad for TTM including, in some embodiments, a multilayered pad body. The pad body includes a conformable layer, a conduit layer over the conformable layer, and an insulation layer over the conduit layer. The conformable layer includes a conformable material configured to conform around a limb of a patient. The conduit layer includes one or more conduits configured to convey a fluid through the conduit layer. The insulation layer is configured to insulate the conduit layer from an ambient temperature of an environment around the patient.

In some embodiments, the pad further includes an inflatable cuff around the pad body configured to press the pad body against the limb of the patient when the inflatable cuff is inflated.

In some embodiments, the pad body further includes an inner impermeable film between the conduit layer and the conformable layer. The inner impermeable film is configured to retain the fluid in the conduit layer.

In some embodiments, the pad body further includes an outer impermeable film between the conduit layer and the insulation layer. The outer impermeable film is configured to retain the fluid in the conduit layer.

In some embodiments, a thermal conductivity of the conformable material increases as the conformable layer conforms around the limb of the patient. The thermal conductivity increases in accordance with collapsing cells of the conformable material, which puts opposite sides of the cells in contact with each other.

In some embodiments, the inflatable cuff is inseparably coupled to the pad body.

In some embodiments, the inflatable cuff is separably coupled to the pad body in a ready-to-use state of the pad.

In some embodiments, the inflatable cuff includes a valve. The valve is configured for inflating or deflating the inflatable cuff

In some embodiments, the pad further includes one or more inlets. The one-or-more inlets are configured for charging the conduit layer with the fluid.

In some embodiments, the pad further includes one or more outlets. The one-or-more outlets are configured for discharging the fluid from the conduit layer.

In some embodiments, the conformable layer is of a sealed sleeve around the conformable material, the sealed sleeve configured to conform the conformable layer around the limb of the patient when air is removed from the conformable material.

In some embodiments, the pad body further includes an outer impermeable film between the conduit layer and the insulation layer. The outer impermeable film is configured to retain the fluid in the conduit layer.

In some embodiments, a thermal conductivity of the conformable material increases as the conformable layer conforms around the limb of the patient. The thermal conductivity increases in accordance with collapsing cells of the conformable material, which puts opposite sides of the cells in contact with each other.

In some embodiments, the sleeve includes a valve. The valve is configured for removing the air from the conformable material in the sleeve.

In some embodiments, the pad further includes one or more inlets. The one-or-more inlets are configured for charging the conduit layer with the fluid.

In some embodiments, the pad further includes one or more outlets. The one-or-more outlets are configured for discharging the fluid from the conduit layer.

Also disclosed herein is a pad for TTM including, in some embodiments, a multilayered pad body a backing over the pad body. The pad body includes an expandable layer, a conduit layer under the expandable layer, and an insulation layer under the conduit layer. The expandable layer includes an expandable composition. The expandable composition is configured to expand and conform around a limb of a patient placed on or disposed in the expandable composition. The conduit layer includes one or more conduits configured to convey a fluid through the conduit layer. The insulation layer is configured to insulate the conduit layer from an ambient temperature of an environment around the patient. The backing over the pad body is over the expandable layer. The backing is configured to be removed before the limb of the patient is placed on or disposed in the expandable composition.

In some embodiments, the pad body further includes an inner impermeable film between the conduit layer and the expandable layer. The inner impermeable film is configured to retain the fluid in the conduit layer.

In some embodiments, the pad body further includes an outer impermeable film between the conduit layer and the insulation layer. The outer impermeable film is configured to retain the fluid in the conduit layer.

In some embodiments, the expandable composition is configured to expand and conform around the limb of the patient when the expandable composition is exposed to air upon removal of the backing.

In some embodiments, the expandable composition is configured to expand and conform around the limb of the patient when the expandable composition is treated with an activating agent.

In some embodiments, the backing is configured to release the activating agent with removal of the backing.

In some embodiments, the pad further includes one or more inlets. The one-or-more inlets are configured for charging the conduit layer with the fluid.

In some embodiments, the pad further includes one or more outlets. The one-or-more outlets are configured for discharging the fluid from the conduit layer.

Also disclosed herein is a pad for TTM including, in some embodiments, a multilayered pad cover and a plurality of thermally conductive beads. The pad cover includes a flexible outer layer and an impermeable film. The impermeable film is between the outer layer and an enclosed space enclosed by the pad cover. The impermeable film is configured to retain a fluid conveyed through the enclosed space. The beads are loosely packed in the enclosed space to allow the fluid to flow around the beads. The pad cover and the beads are configured to conform around a limb or body of a patient placed thereon.

In some embodiments, the pad further includes one or more inlets. The one-or-more inlets are configured for charging the enclosed space with the fluid.

In some embodiments, the further includes one or more outlets. The one-or-more outlets are configured for discharging the fluid from the conduit layer.

In some embodiments, the pad is configured for placement over a mattress of a hospital bed.

In some embodiments, the pad is configured as a mattress of a hospital bed.

Also disclosed herein is a system for TTM including, in some embodiments, a control module and a pad selected from the foregoing pads having the one-or-more inlets and the one-or-more outlets of the pad. The control module includes a hydraulic system including a chiller evaporator, one or more outlets, and one or more inlets. The chiller evaporator is configured for chilling a fluid to produce a chilled fluid. The one-or-more outlets of the hydraulic system are configured for discharging the chilled fluid from the hydraulic system. The one-or-more inlets of the hydraulic system are configured for charging the hydraulic system with the fluid to continue to produce the chilled fluid. The pad is configured to convey the chilled fluid through the pad. The one-or-more inlets of the pad are configured for charging the pad with the chilled fluid. The one-or-more outlets of the pad are configured for discharging the fluid from the pad.

Also disclosed herein is a method of a system for TTM including, in some embodiments, a connecting step, a patient-placing step, a conforming step, and a therapeutic hypothermia-inducing step. The connecting step includes connecting one or more inlets and one or more outlets of the pad to a hydraulic system of a control module. The pad includes a multilayered pad body having a conduit layer or a multilayered pad cover having an enclosed space configured to convey a fluid. The patient-placing step includes placing a limb or body of a patient on the pad. The conforming step includes causing the pad to conform to the limb or body of the patient. The therapeutic hypothermia-inducing step includes inducing therapeutic hypothermia in the patient by circulating a chilled fluid provided by the control module through the conduit layer or the enclosed space of the pad.

In some embodiments, the conforming step includes inflating an inflatable cuff around the pad body. The pad body includes a conformable layer of a conformable material, the conduit layer over the conformable layer, and an insulation layer over the conduit layer.

In some embodiments, the conforming step includes removing air from a sealed sleeve of the pad body. The pad body includes a conformable material within the sleeve, the conduit layer over a conformable layer including the sleeve around the conformable material, and an insulation layer over the conduit layer.

In some embodiments, a thermal conductivity of the conformable material increases as the conformable layer conforms around the limb or body of the patient. The thermal conductivity increases in accordance with collapsing cells of the conformable material and putting opposite sides of the cells in contact with each other.

In some embodiments, the conforming step includes removing a backing over the pad body before placing the limb or body of the patient on the pad. The pad body includes an expandable layer including an expandable composition, the conduit layer under the expandable layer, and an insulation layer under the conduit layer.

In some embodiments, removing the backing over the pad body exposes the expandable composition to air, which causes the expandable composition to conform around the limb or body of the patient.

In some embodiments, removing the backing over the pad body releases an activating agent for the expandable composition, which causes the expandable composition to conform around the limb or body of the patient.

In some embodiments, the patient-placing step causes the conforming step. The pad cover includes a flexible outer layer over an impermeable film enclosing the enclosed space. The enclosed space has therein a plurality of thermally conductive beads loosely packed in the enclosed space.

In some embodiments, the connecting step includes connecting a single, dedicated inlet of the one-or more inlets to an outlet of the hydraulic system of the control module.

In some embodiments, the connecting step includes connecting a single, dedicated outlet of the one-or-more outlets to an inlet of the hydraulic system of the control module.

In some embodiments, the method further includes a pad-placing step. The pad-placing step includes placing the pad over a mattress of a hospital bed before the patient-placing step.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2 illustrates a first pad for TTM in accordance with some embodiments.

FIG. 3 illustrates a cross-section of the first pad in accordance with some embodiments.

FIG. 4 illustrates a second pad for TTM in accordance with some embodiments.

FIG. 5 illustrates a cross-section of the second pad in accordance with some embodiments.

FIG. 6 illustrates a third pad for TTM in accordance with some embodiments.

FIG. 7 illustrates a cross-section of the third pad in accordance with some embodiments.

FIG. 8 illustrates a fourth pad for TTM in accordance with some embodiments.

FIG. 9 illustrates a cross-section of the fourth pad in accordance with some embodiments.

FIG. 10 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, current systems for TTM require adhesive pads adhered onto patients in order to maintain sufficient contact between the pads and the patients for inducing therapeutic hypothermia by circulating chilled fluid through the pads. However, such pads can cause skin irritation from the adhesive of the pads. In addition, clinicians often require open access to the patients for medical procedures. The adhesive pads can hinder the open access required by the clinicians.

Disclosed herein are TTM systems, pads, and methods thereof that address the foregoing.

### TTM Systems

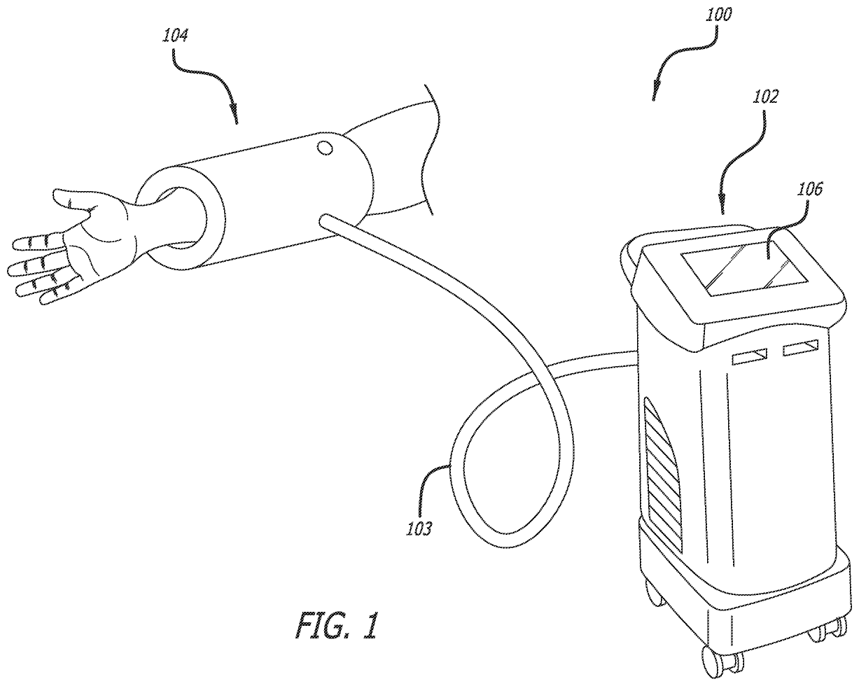

FIG. 1 illustrates a TTM system 100 in accordance with some embodiments.

As shown, the system 100 includes a TTM control module 102, a TTM pad such as the first pad 104 set forth below, and one or more fluid conduits 103 therebetween. Description for the control module 102 is set forth immediately below. Description for several embodiments of the TTM pad is set forth in a following section.

The control module 102 includes a console 106 with an integrated display screen configured as a touchscreen for operating the control module 102. The console 106 includes one or more processors, primary and secondary memory, and instructions stored in the primary memory configured to instantiate one or more processes for TTM with the control module 102.

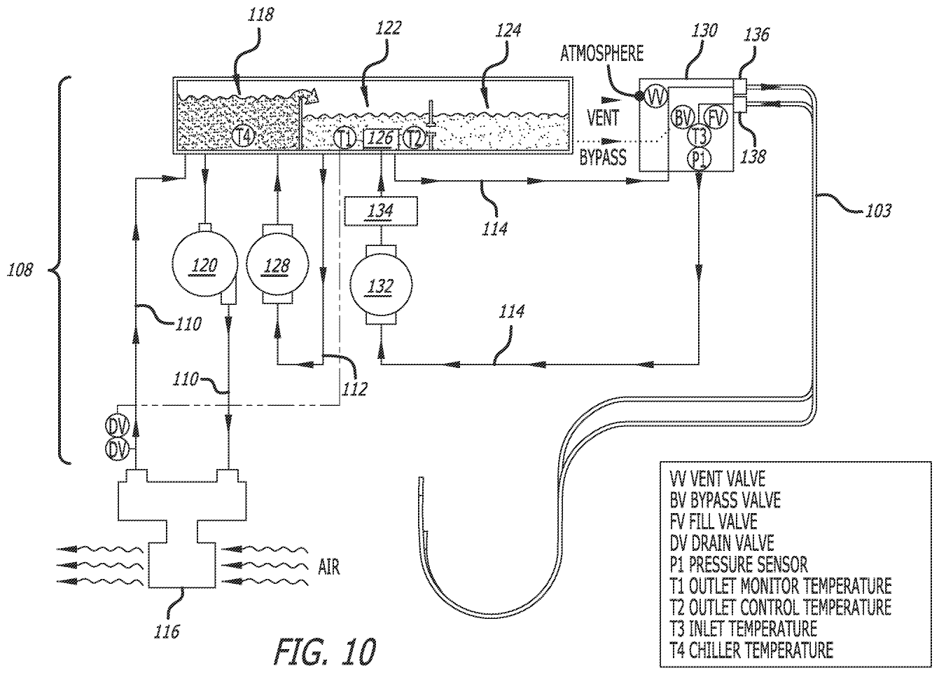

FIG. 10 illustrates a hydraulic system 108 of the control module 102 in accordance with some embodiments.

The control module 102 also includes the hydraulic system 108, which includes a chiller circuit 110, a mixing circuit 112, and a circulating circuit 114.

The chiller circuit 110 is configured for chilling a fluid (e.g., water, ethylene glycol, a combination of water and ethylene glycol, etc.) to produce a chilled fluid, which chilled fluid, in turn, is for mixing with an optionally heated fluid to produce a supply fluid for TTM. The chiller circuit 110 includes a chiller evaporator 116 configured for the chilling of the fluid passing therethrough. The fluid for the chilling by the chiller evaporator 116 is provided by a chiller tank 118 using a chiller pump 120 of the chiller circuit 110.

The mixing circuit 112 is configured for mixing spillover of the chilled fluid from the chiller tank 118 with a mixed fluid in a mixing tank 122 of the mixing circuit 112. The mixing circuit 112 includes a heater 126 in the mixing tank 122 configured for heating the mixed fluid to produce a heated fluid if needed for mixing with the chilled fluid to provide a supply tank 124 of the circulating circuit 114 with a supply fluid of a desired temperature for TTM. The mixing circuit 112 includes a mixing pump 128 configured to pump the fluid from the mixing tank 122 into the chiller tank 118 for producing the chilled fluid and the spillover of the chilled fluid for the mixing tank 122.

The circulating circuit 114 is configured for circulating the supply fluid for TTM, which includes circulating the supply fluid provided by the manifold 130 through the TTM pad using a circulation pump 132 directly or indirectly governed by a flow meter 134 of the circulating circuit 114. The manifold 130 includes one or more outlets 136 configured for discharging the supply fluid (e.g., a chilled fluid) from the hydraulic system 108 and one or more inlets 138 configured for charging the hydraulic system 108 with return fluid from the TTM pad to continue to produce the supply fluid.

### TTM Pads

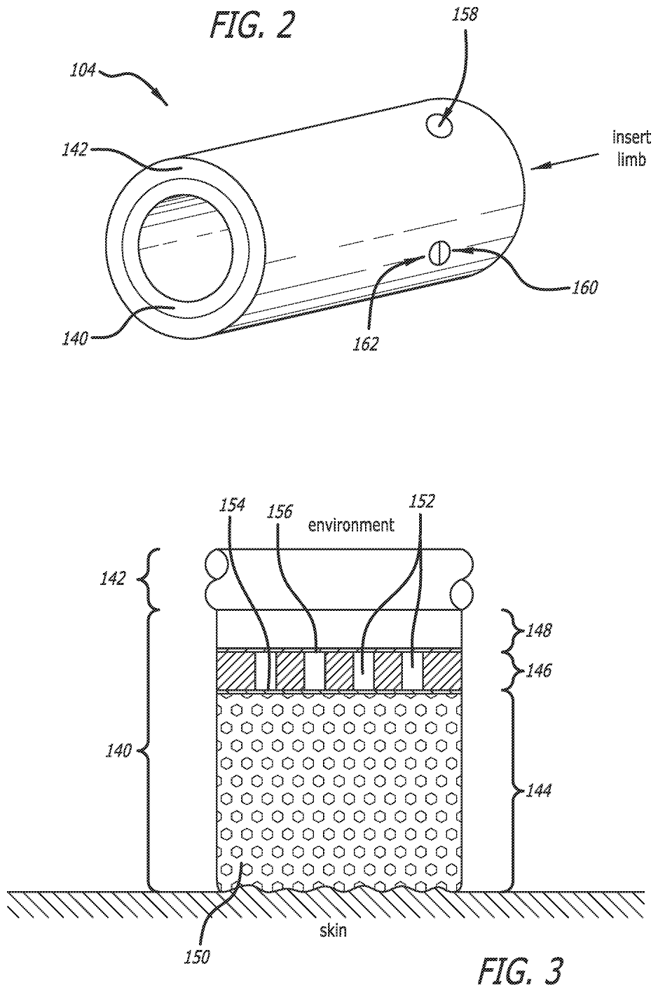

FIG. 2 illustrates the first pad 104 in accordance with some embodiments. FIG. 3 illustrates a cross-section of the pad 104 in accordance with some embodiments.

As shown, the pad 104 includes a multilayered pad body 140 and an inflatable cuff 142 around the pad body 140. While description of the pad 104 set forth below is directed to its configuration for a limb of a patient, the pad 104 can alternatively be configured for a body (e.g., a torso) of the patient.

The pad body 140 includes a conformable layer 144, a conduit layer 146 over the conformable layer 144, and an insulation layer 148 over the conduit layer 146.

The conformable layer 144 includes a conformable material 150 (e.g., a viscoelastic or low-resilience polyurethane foam) configured to conform around the limb of the patient. A thermal conductivity of the conformable material 150 increases as the conformable layer 144 conforms around the limb of the patient. The thermal conductivity increases in accordance with collapsing cells of the conformable material 150, which puts opposite sides of the cells in contact with each other. In addition, the collapsing of the cells expels air from the cells, which further increase the thermal conductivity of the conformable material 150 as air has a poor thermal conductivity.

The conduit layer 146 includes one or more conduits 152 configured to convey a fluid (e.g., the chilled fluid) through the conduit layer 146. The one-or-more conduits 152 can be one-or-more channels formed in a material of the conduit layer 146, or the one-or-more conduits 152 can be one-or-more tubes disposed in the material of the conduit layer 146.

The insulation layer 148 is configured to insulate the conduit layer 146 from an ambient temperature of an environment around the patient. The insulation layer 148 can be a same material as the conformable material 150 or a different material than the conformable material 150.

The pad body 140 can further include an inner impermeable film 154 between the conduit layer 146 and the conformable layer 144, an outer impermeable film 156 between the conduit layer 146 and the insulation layer 148, or both the inner impermeable film 154 and the outer impermeable film 156. Each impermeable film of the inner impermeable film 154 and the outer impermeable film 156 is configured to retain the fluid in the conduit layer 146, particularly if the one-or-more conduits 152 are the one-or-more channels formed in the material of the conduit layer 146. Even if the one-or-more conduits 152 are the one-or-more tubes disposed in the material of the conduit layer 146, the inner impermeable film 154 or the outer impermeable film 156 can provide secondary containment.

The inflatable cuff 142 can be inseparably coupled to the pad body 140 or separably coupled to the pad body in a ready-to-use state of the pad. The inflatable cuff 142 is configured to press the pad body 140 against the limb of the patient when the inflatable cuff 142 is inflated by way of a valve 158 configured for inflating or deflating the inflatable cuff 142. While not shown, the inflatable cuff 142 can include a captive buckle frame through which a loose end of a strap attached to the inflatable cuff 142 having opposing hook-and-loop-fastener faces is configured to pass and fold over onto itself for loosely securing the pad 104 to the limb of the patient before the inflatable cuff 142 is inflated.

In addition to the foregoing, the pad 104 further includes one or more inlets 160 and one or more outlets 162. The one-or-more inlets 160 are configured for charging the conduit layer 146 with the fluid, while the one-or-more outlets 162 are configured for discharging the fluid from the conduit layer 146.

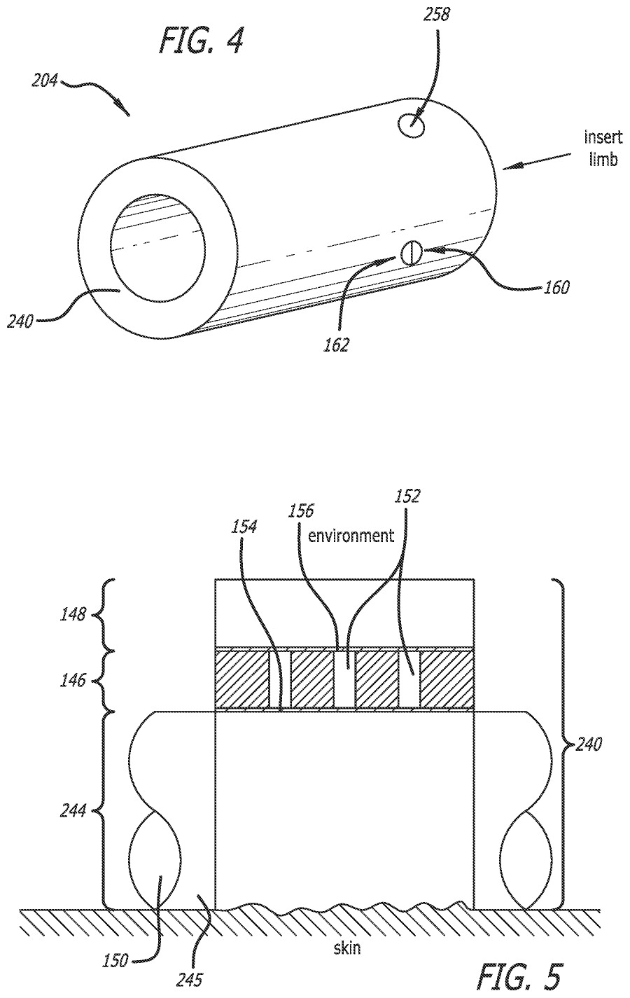

FIG. 4 illustrates a second pad 204 for TTM in accordance with some embodiments. FIG. 5 illustrates a cross-section of the second pad 204 in accordance with some embodiments.

As shown, the pad 204 includes a multilayered pad body 240. The pad body 240 includes a conformable layer 244, the conduit layer 146 over the conformable layer 244, and the insulation layer 148 over the conduit layer 146. While description of the pad 204 set forth below is directed to its configuration for a limb of a patient, the pad 204 can alternatively be configured for a body (e.g., a torso) of the patient.

The conformable layer 244 includes a sealed sleeve 245 around the conformable material 150. The sleeve 245 is configured to conform the conformable layer 244 around the limb of the patient when air is removed from the conformable material 150 in the sleeve 245 by way of a valve 258, which valve is configured for removing the air from the conformable material 150 in the sleeve 245. While not shown, the sleeve 245 can include a captive buckle frame through which a loose end of a strap attached to the sleeve 245 having opposing hook-and-loop-fastener faces is configured to pass and fold over onto itself for loosely securing the pad 204 to the limb of the patient before air is removed from the sleeve 245. As set forth above, the conformable material 150 can be a viscoelastic or low-resilience polyurethane foam, the thermal conductivity of which increases as the conformable layer 244 conforms around the limb of the patient. The thermal conductivity increases in accordance with collapsing cells of the conformable material 150, which puts opposite sides of the cells in contact with each other as well as expels air from the cells.

As set forth above, the conduit layer 146 includes the one-or-more conduits 152 configured to convey a fluid (e.g., the chilled fluid) through the conduit layer 146. The one-or-more conduits 152 can be one-or-more channels formed in a material of the conduit layer 146, or the one-or-more conduits 152 can be one-or-more tubes disposed in the material of the conduit layer 146.

As set forth above, the insulation layer 148 is configured to insulate the conduit layer 146 from an ambient temperature of an environment around the patient. The insulation layer 148 can be a same material as the conformable material 150 or a different material than the conformable material 150.

The pad body 240 can further include the inner impermeable film 154 between the conduit layer 146 and the conformable layer 244, the outer impermeable film 156 between the conduit layer 146 and the insulation layer 148, or both the inner impermeable film 154 and the outer impermeable film 156. Each impermeable film of the inner impermeable film 154 and the outer impermeable film 156 is configured to retain the fluid in the conduit layer 146, particularly if the one-or-more conduits 152 are the one-or-more channels formed in the material of the conduit layer 146. Even if the one-or-more conduits 152 are the one-or-more tubes disposed in the material of the conduit layer 146, the inner impermeable film 154 or the outer impermeable film 156 can provide secondary containment.

In addition to the foregoing, the pad 204 further includes the one-or-more inlets 160 and the one-or-more outlets 162. The one-or-more inlets 160 are configured for charging the conduit layer 146 with the fluid, while the one-or-more outlets 162 are configured for discharging the fluid from the conduit layer 146.

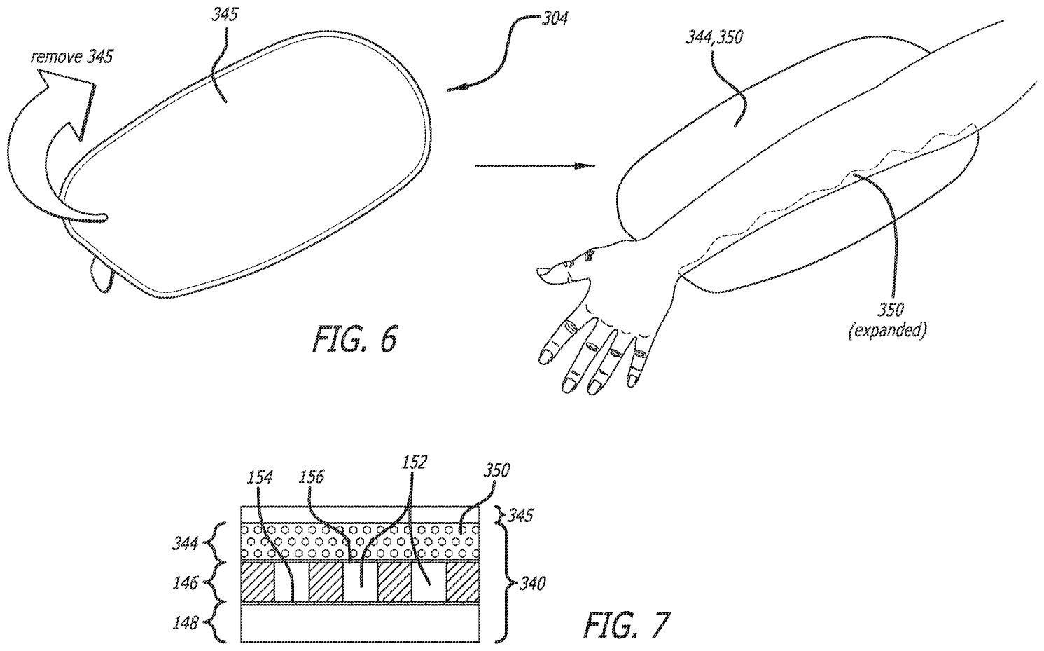

FIG. 6 illustrates a third pad 304 for TTM in accordance with some embodiments. FIG. 7 illustrates a cross-section of the third pad 304 in accordance with some embodiments.

As shown, the pad 304 includes a multilayered pad body 340 and a backing 345 over the pad body 340. While description of the pad 304 set forth below is directed to its configuration for a limb of a patient, the pad 304 can alternatively be configured for a body (e.g., a torso) of the patient.

The pad body 340 includes an expandable layer 344, the conduit layer 146 under the expandable layer 344, and the insulation layer 148 under the conduit layer 146.

The expandable layer 344 includes an expandable composition 350. The expandable composition 350 is configured to expand and conform around the limb of the patient placed on or disposed in the expandable composition 350. The expandable composition 350 is configured to expand and conform around the limb of the patient when the expandable composition 350 is exposed to air. Alternatively, the expandable composition 350 is configured to expand and conform around the limb of the patient when the expandable composition 350 is treated with an activating agent. The backing 345 is configured to be removed before the limb of the patient is placed on or disposed in the expandable composition 350. Indeed, since the backing 345 is over the expandable layer 344 of the pad body 340, removal of the backing 345 exposes the expandable composition 350 to air for expansion thereof. Alternatively, the backing 345 can be configured to release an activating agent upon removal of the backing 345 for expansion of the expandable composition 350.

As set forth above, the conduit layer 146 includes the one-or-more conduits 152 configured to convey a fluid (e.g., the chilled fluid) through the conduit layer 146. The one-or-more conduits 152 can be one-or-more channels formed in a material of the conduit layer 146, or the one-or-more conduits 152 can be one-or-more tubes disposed in the material of the conduit layer 146.

As set forth above, the insulation layer 148 is configured to insulate the conduit layer 146 from an ambient temperature of an environment around the patient. The insulation layer 148 can be a same material as the conformable material 150 or a different material than the conformable material 150.

The pad body 340 can further include the inner impermeable film 154 between the conduit layer 146 and the conformable layer 244, the outer impermeable film 156 between the conduit layer 146 and the insulation layer 148, or both the inner impermeable film 154 and the outer impermeable film 156. Each impermeable film of the inner impermeable film 154 and the outer impermeable film 156 is configured to retain the fluid in the conduit layer 146, particularly if the one-or-more conduits 152 are the one-or-more channels formed in the material of the conduit layer 146. Even if the one-or-more conduits 152 are the one-or-more tubes disposed in the material of the conduit layer 146, the inner impermeable film 154 or the outer impermeable film 156 can provide secondary containment.

In addition to the foregoing, the pad 304 further includes the one-or-more inlets 160 and the one-or-more outlets 162. The one-or-more inlets 160 are configured for charging the conduit layer 146 with the fluid, while the one-or-more outlets 162 are configured for discharging the fluid from the conduit layer 146.

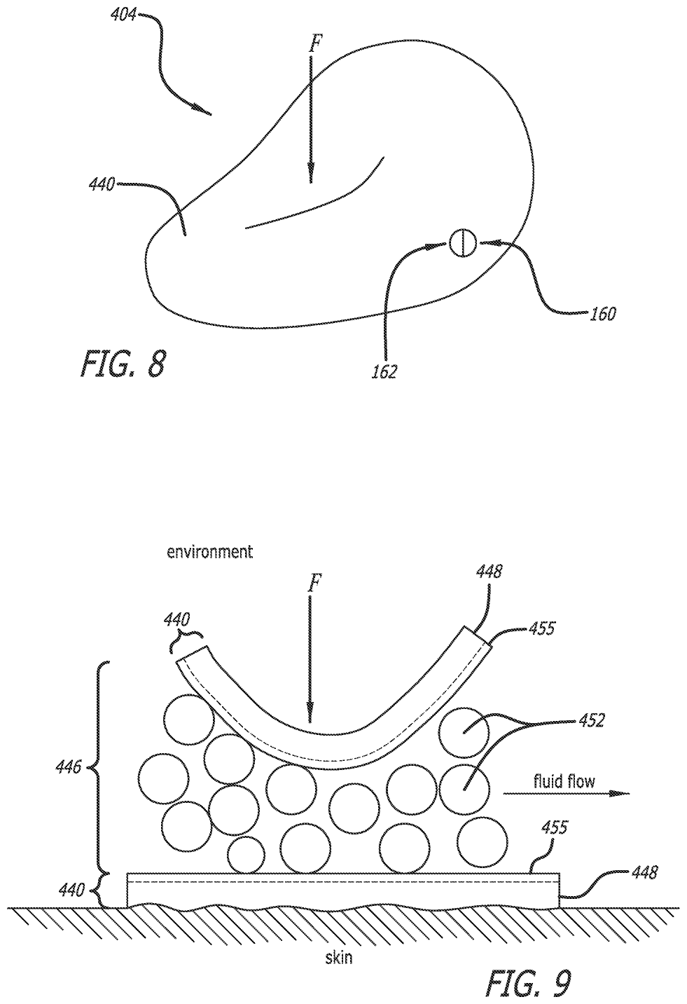

FIG. 8 illustrates a fourth pad 404 for TTM in accordance with some embodiments. FIG. 9 illustrates a cross-section of the fourth pad 404 in accordance with some embodiments.

As shown, the pad 404 includes a multilayered pad cover 440 and a plurality of thermally conductive beads 452 within an enclosed space 446 enclosed by the pad cover 440. The pad cover 440 and the beads 452 are configured to conform around a limb or body of a patient placed thereon. Therefore, the pad 404 can be configured for placement over a mattress of a hospital bed such as a portion of the mattress for the limb of the patient or an entirety of the mattress for the body of the patient. Alternatively, the pad 404 is configured as a mattress of such a hospital bed.

The pad cover 440 includes a flexible outer layer 448 and an optional impermeable film 455.

The outer layer 448 can be a relatively thin layer of the conformable material 150, a breathable fabric, or the thin layer of the conformable material 150 and the breathable fabric thereover. While not shown, the outer layer 448 can include a number of outward-facing protrusions formed of the conformable material 150 or another conformable material configured to press against skin of the limb or body of the patient placed on the pad 404. The protrusions disturb flow of a fluid through the enclosed space 446, thereby causing turbulent that is effective for convective heat transfer while minimizing fluidic pressure drop.

The impermeable film 455, when present, is between the outer layer 448 and the enclosed space 446 enclosed by the pad cover 440. The impermeable film 455 is configured to retain a fluid conveyed through the enclosed space 446.

The beads 452 are of a highly thermally conductible material, and the beads 452 loosely packed in the enclosed space 446 to allow the fluid to flow in a flow path around the beads 452. The pad cover 440 and the beads 452 are configured to conform around the limb or body of the patient placed thereon, so the flow path of the fluid around the beads 452 is contingent with a position of the limb or body of the patient on the pad 404. Advantageously, the pad 404 and the flow path of the fluid therethrough can continuously adapt as the patient moves on the pad. (See, for example, F in FIGS. 8 and 9.)

In addition to the foregoing, the pad 404 further includes the one-or-more inlets 160 and the one-or-more outlets 162. The one-or-more inlets 160 are configured for charging the enclosed space 446 with the fluid, while the one-or-more outlets 162 are configured for discharging the fluid from the enclosed space 446.

### Methods

Methods of the systems and pads include methods of use. For example, a method of using the system 100 includes a connecting step, a patient-placing step, a conforming step, and a therapeutic hypothermia-inducing step.

The method can further include a pad-placing step if the pad 104, 204, 304, or 404 is not already in place before the patient-placing step. The pad-placing step includes placing the pad 104, 204, 304, or 404 over a mattress of a hospital bed.

The connecting step includes connecting the one-or-more inlets 160 and the one-or-more outlets 162 of the pad 104, 204, 304, or 404 to the hydraulic system 108 of the control module 102. The connecting step includes connecting a single, dedicated inlet of the one-or-more inlets 160 of the pad 104, 204, 304, or 404 to an outlet of the one-or-more outlets 136 of the hydraulic system 108 of the control module 102, as well as connecting a single, dedicated outlet of the one-or-more outlets 162 of the pad 104, 204, 304, or 404 to an inlet of the one-or-more inlets 138 of the hydraulic system 108 of the control module 102.

The patient-placing step includes placing a limb of a patient in the pad 104 or 204, the limb of the patient on the pad 304 or 404, or a body of the patient on the pad 304 or 404.

The conforming step includes causing the pad 104 or 204 to conform to the limb or body of the patient. As to the pad 104, causing the pad 104 or 204 to conform to the limb or body of the patient includes inflating the inflatable cuff 142 around the pad body 140. As set forth above, the pad body 140 includes the conformable layer 144 of the conformable material 150, the conduit layer 146 over the conformable layer 144, and the insulation layer 148 over the conduit layer 146. As to the pad 204, causing the pad 104 or 204 to conform to the limb or body of the patient includes removing air from the sleeve 245 of the pad body 240. As set forth above, the pad body 240 includes the conformable material 150 within the sleeve 245, the conduit layer 146 over the conformable 244 layer including the sleeve 245 around the conformable material 150, and the insulation layer 148 over the conduit layer 146. Again, the thermal conductivity of the conformable material 150 increases as the conformable layer 144 or 244 conforms around the limb or body of the patient. The thermal conductivity increases in accordance with collapsing cells of the conformable material 150 and putting opposite sides of the cells in contact with each other while expelling air from the cells.

Alternatively, the conforming step includes causing the pad 304 to conform to the limb or body of the patient. Causing the pad 304 to conform to the limb or body of the patient includes removing the backing 345 over the pad body 340 before placing the limb or body of the patient on the pad 304. As set forth above, the pad body 340 includes the expandable layer 344 including an expandable composition 350, the conduit layer 146 under the expandable layer 344, and the insulation layer 148 under the conduit layer. Removing the backing 345 over the pad body 340 exposes the expandable composition 350 to air, which can cause the expandable composition 350 to conform around the limb or body of the patient when so configured. Alternatively, removing the backing 345 over the pad body 340 releases an activating agent for the expandable composition 350, which causes the expandable composition 350 to conform around the limb or body of the patient when so configured.

Alternatively, the conforming step includes causing the pad 404 to conform to the limb or body of the patient. Causing the pad 404 to conform to the limb or body of the patient includes merely practicing the patient-placing step with the pad 404, as the patient-placing step causes the conforming step. As set forth above, the pad cover 440 includes the flexible outer layer 448 over the impermeable film 455 enclosing the enclosed space 446. The enclosed space 446 has therein the plurality of thermally conductive beads 452 loosely packed in the enclosed space 446. As such, both the pad cover 440 with the flexible outer layer 448 and the beads 452 loosely pack in the enclosed space 446 enable the pad 404 to conform around the limb or body of the patient when placed thereon.

The therapeutic hypothermia-inducing step includes inducing therapeutic hypothermia in the patient by circulating the supply fluid (e.g., a chilled fluid) provided by the control module 102 through the conduit layer 146 of the pad 104, 204, or 304 or the enclosed space 446 or the pad 404.

The method can further include a temperature-returning step of returning the patient to his or her normal body temperature by way of the pad 104, 204, 304, or 404. Alternatively, the method set forth above does not include the therapeutic hypothermia-inducing step but instead includes a temperature-maintaining step of maintaining the patient at his or her normal body temperature to combat fever or neurological symptoms.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A pad for targeted temperature management (“TTM”), comprising:
a multilayered pad body including:
a conformable layer including a conformable material configured to conform around a limb of a patient;
a conduit layer over the conformable layer including one or more conduits configured to convey a fluid through the conduit layer;
an insulation layer over the conduit layer configured to insulate the conduit layer from an ambient temperature of an environment around the patient.

2. The pad of claim 1, further comprising an inflatable cuff around the pad body configured to press the pad body against the limb of the patient when the inflatable cuff is inflated.

3. The pad of claim 2, the pad body further including an inner impermeable film between the conduit layer and the conformable layer configured to retain the fluid in the conduit layer.

4. The pad of claim 2, the pad body further including an outer impermeable film between the conduit layer and the insulation layer configured to retain the fluid in the conduit layer.

5. The pad of claim 2, wherein a thermal conductivity of the conformable material increases as the conformable layer conforms around the limb of the patient, the thermal conductivity increasing in accordance with collapsing cells of the conformable material and putting opposite sides of the cells in contact with each other.

6. The pad of claim 2, wherein the inflatable cuff is inseparably coupled to the pad body.

7. The pad of claim 2, wherein the inflatable cuff is separably coupled to the pad body in a ready-to-use state of the pad.

8. The pad of claim 2, wherein the inflatable cuff includes a valve configured for inflating or deflating the inflatable cuff.

9. The pad of claim 2, further comprising one or more inlets configured for charging the conduit layer with the fluid.

10. The pad of claim 9, further comprising one or more outlets configured for discharging the fluid from the conduit layer.

11. The pad of claim 1, wherein the conformable layer is of a sealed sleeve around the conformable material, the sealed sleeve configured to conform the conformable layer around the limb of the patient when air is removed from the conformable material.

12. The pad of claim 11, the pad body further including an outer impermeable film between the conduit layer and the insulation layer configured to retain the fluid in the conduit layer.

13. The pad of claim 11, wherein a thermal conductivity of the conformable material increases as the conformable layer conforms around the limb of the patient, the thermal conductivity increasing in accordance with collapsing cells of the conformable material and putting opposite sides of the cells in contact with each other.

14. The pad of claim 11, wherein the sleeve includes a valve configured for removing the air from the conformable material in the sleeve.

15. The pad of claim 11, further comprising one or more inlets configured for charging the conduit layer with the fluid.

16. The pad of claim 15, further comprising one or more outlets configured for discharging the fluid from the conduit layer.

17. A pad for targeted temperature management (“TTM”), comprising:
a multilayered pad body including:
an expandable layer including an expandable composition configured to expand and conform around a limb of a patient placed on or disposed in the expandable composition;
a conduit layer under the expandable layer including one or more conduits configured to convey a fluid through the conduit layer; and
an insulation layer under the conduit layer configured to insulate the conduit layer from an ambient temperature of an environment around the patient; and
a backing over the expandable layer configured to be removed before the limb of the patient is placed on or disposed in the expandable composition.

18. The pad of claim 17, the pad body further including an inner impermeable film between the conduit layer and the expandable layer configured to retain the fluid in the conduit layer.

19. The pad of claim 17, the pad body further including an outer impermeable film between the conduit layer and the insulation layer configured to retain the fluid in the conduit layer.

20. The pad of claim 17, wherein the expandable composition is configured to expand and conform around the limb of the patient when the expandable composition is exposed to air upon removal of the backing.

21. The pad of claim 17, wherein the expandable composition is configured to expand and conform around the limb of the patient when the expandable composition is treated with an activating agent.

22. The pad of claim 21, wherein the backing is configured to release the activating agent with removal of the backing.

23. The pad of claim 17, further comprising one or more inlets configured for charging the conduit layer with the fluid.

24. The pad of claim 23, further comprising one or more outlets configured for discharging the fluid from the conduit layer.

25. A pad for targeted temperature management (“TTM”), comprising:
a multilayered pad cover including:
a flexible outer layer; and
an impermeable film between the outer layer and an enclosed space enclosed by the pad cover, the impermeable film configured to retain a fluid conveyed through the enclosed space; and
a plurality of thermally conductive beads loosely packed in the enclosed space to allow the fluid to flow around the beads, the pad cover and the beads configured to conform around a limb or body of a patient placed thereon.

26. The pad of claim 25, further comprising one or more inlets configured for charging the enclosed space with the fluid.

27. The pad of claim 26, further comprising one or more outlets configured for discharging the fluid from the conduit layer.

28. The pad of claim 25, wherein the pad is configured for placement over a mattress of a hospital bed.

29. The pad of claim 25, wherein the pad is configured as a mattress of a hospital bed.
